(19) United States
(12) Patent Application Publication
(10) Pub. No.: US 2005/0112620 A1
(43) Pub. Date: May 26, 2005

(54) Nucleic acid mapping using linear analysis

(75) Inventor: Eugene Y. Chan, Boston, MA (US)

Correspondence Address: Maria A. Trevisan;Wolf, Greenfield & Sacks, P.C., 600 Atlantic Avenue, Boston, MA, 02210, US

(73) Assignee: U.S. Genomics, Inc., Woburn, MA (US)

(21) Appl. No.: 10/910,253

(22) Filed: Aug. 2, 2004

Related U.S. Application Data
(60) Provisional application No. 60/492,376, filed on Aug. 4, 2003.

(51) Int. Cl. C12Q 1/68; C12P 19/34

(52) U.S. Cl. 435/6; 435/91.2

(57) ABSTRACT

The invention relates to the use of nucleic acid binding agents for labeling polymers such as nucleic acid molecules. The nucleic acid binding agents are nucleic acid binding proteins that bind nucleic acid molecules non-specifically, in some embodiments.

### RELATED APPLICATIONS

This application claims priority under 35 U.S.C. §119 to U.S. Provisional Patent Application Ser. No. 60/492,376, filed Aug. 4, 2003, the entire contents of which are hereby incorporated by reference.

### FIELD OF THE INVENTION

The invention provides new compositions and methods of use thereof for labeling and analyzing nucleic acid molecules.

### BACKGROUND OF THE INVENTION

Many technologies relating to genomic sequencing and analysis require time- and labor-intensive steps. Current approaches to transposon mapping, for instance, are tedious, cumbersome and rely on time-intensive steps such as PCR, and Sanger sequencing. These methods are challenging. Global mutation analysis using these methods to understand genome function often requires year to perform because of the iterative nature of the approach. Footprinting analysis also requires many tedious steps and generally must be performed on small pieces of DNA.

### SUMMARY OF THE INVENTION

The methods of the invention involve improved methods for analyzing nucleic acids using linear analysis techniques. In one aspect the invention relates to a method for identifying a region of a nucleic acid by protecting one or more regions of a nucleic acid with a protective compound, contacting the protected nucleic acid with a blocking compound to block the non-protected regions of the nucleic acid, removing the protective compound, and contacting the nucleic acid with a first label, wherein the first label is detectably distinct from the blocking compound, and detecting the position of the first label on the nucleic acid to identify the region of the nucleic acid with a linear nucleic acid analysis system. Regions of the nucleic acid that are protected by the protective compound are usually those regions that are also labeled with the first label. As used herein, to protect a region of the nucleic acid means to prevent that region from interacting with the blocking compound. As used herein, to block a region of the nucleic acid means to prevent that region from interacting with the first label.

In one embodiment the blocking compound is a second label and is optionally a fluorescent label.

In another embodiment the protective compound is a RecA filament. In yet other embodiments the protective compound is a protein, an oligonucleotide, a peptide nucleic acid (PNA), a locked nucleic acid (LNA), a DNA, an RNA, a bisPNA clamp, a pseudocomplementary PNA, or a LNA-DNA co-polymer. Optionally the protective compound is an enzyme, such as a DNA polymerase, an RNA polymerase, a DNA repair enzyme, a helicase, a nuclease, or a ligase. The protective compound may bind to the nucleic acid in a sequence specific or a sequence non-specific manner.

The first label may be a fluorescent label. In some embodiments the first label is a backbone specific label. In other embodiments the first label is selected from the group consisting of an electron spin resonance molecule, a fluorescent molecule, a chemiluminescent molecule, a radioisotope, an enzyme substrate, a biotin molecule, an avidin molecule, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

The nucleic acid is DNA or RNA in some embodiments.

A method for determining a property of a nucleic acid-protein interaction is provided according to another aspect of the invention. The method involves contacting a first nucleic acid with a first protein, determining a first binding interaction between the first nucleic acid and the first protein, and comparing the first binding interaction with a second binding interaction with a linear nucleic acid analysis system to determine the property of the nucleic acid-protein interaction.

In one embodiment the second binding interaction involves contacting a second nucleic acid with a second protein, and determining the second binding interaction between the second nucleic acid and the second protein. The first and second nucleic acid and the first and second protein may be identical, similar, overlapping or different. The step of contacting the first protein with the first nucleic acid may optionally involve the use of a higher concentration of protein relative to nucleic acid than the concentration of protein relative to nucleic acid used in the step of contacting the second protein with the second nucleic acid. Optionally a third nucleic acid is contacted with a third protein and the concentration of protein relative to nucleic acid used in the step of contacting the third protein with the third nucleic acid is higher than the concentration of protein relative to nucleic acid used in the step of contacting the first protein with the first nucleic acid.

In another embodiment the step of contacting the first nucleic acid with the first protein is conducted for a first period of time, and wherein the second binding interaction involves contacting a second nucleic acid identical to the first nucleic acid with a second protein identical to the first protein for a second period of time that is different than the first period of time.

In another embodiment the second binding interaction involves contacting a second nucleic acid identical to the first nucleic acid with a second protein identical to the first protein in the presence of a competitor, which is optionally an oligonucleotide.

The protein, in some embodiments, is a transcription factor. In other embodiments the protein is present in a nuclear extract or a cytoplasmic extract. The protein may bind to the nucleic acid non-specifically or specifically.

In another aspect the invention is a method for identifying a transposon, by scanning a nucleic acid sequence comprising at least one labeled transposon with a linear nucleic acid analysis system to identify the transposon. In one embodiment the transposon includes a tag-site spliced therein. In other embodiments the transposon is an artificial transposon or a natural transposon. In some embodiments multiple transposons are identified within the nucleic acid.

The nucleic acid may be genomic DNA, which optionally is digested prior to linear analysis.

In some embodiments the method involves determining an effect on gene function of the insertion of the transposon. The effect in gene function may be determined, for instance, by assessing gene function in a nucleic acid without a transposon and comparing it with the gene function in the same nucleic acid with a transposon.

In an embodiment the linear nucleic acid analysis system is a single nucleic acid analysis system. In another embodiment the linear nucleic acid analysis system is selected from the group consisting of Gene Engine™, optical mapping, and DNA combing. According to yet another embodiment the linear nucleic acid analysis system comprises exposing the nucleic acid to a station to produce a signal arising from the first label of the nucleic acid or the labeled transposon, and detecting the signal using a detection system.

Each of the limitations of the invention can encompass various embodiments of the invention. It is, therefore, anticipated that each of the limitations of the invention involving any one element or combinations of elements can be included in each aspect of the invention.

### DETAILED DESCRIPTION OF THE INVENTION

The invention involves linear analysis of nucleic acids. The methods are useful for analyzing large nucleic acid segments to identify, for instance, the presence of specific sequences, gene function, genetic mutations, kinetics and other properties of protein-DNA interactions, etc. One method of the invention, for instance, involves footprinting of specific sequences in the genome. The application of the linear nucleic acid analysis technology to the analysis of complex genomes generally involves site-specific labeling of genomic DNA with high efficiency, high specificity, and a large number of fluorescent tags per site. One potential drawback of these approaches for complex genomes is that a limited number of fluorescent labels can be attached to the tags without hindering their ability to bind efficiently to the sequences of interest. The methods of the invention, in some aspects, involve footprinting using a rational site protection strategy as a technique to map specific sequences in the genome. This approach can be applied to a wide range of proteins with linear nucleic acid analysis techniques to map footprinted sites on a target DNA strand of interest.

Thus, in one aspect the invention relates to a method for identifying a region of a nucleic acid by protecting one or more regions of a nucleic acid with a protective compound, contacting the protected nucleic acid with a blocking compound to block the non-protected regions of the nucleic acid, removing the protective compound, and contacting the nucleic acid with a first label, wherein the first label is detectably distinct from the blocking compound, and detecting the position of the first label on the nucleic acid to identify the region of the nucleic acid with a linear nucleic acid analysis system.

A “protective compound” as used herein is any type of compound that binds to a nucleic acid in a sequence specific or non-specific manner. In some embodiments it is preferred that the protective compounds bind to and “protect” specific sequences within a nucleic acid.

The sequence specific protective compounds and/or nucleic acid binding proteins and molecules of the invention (i.e. referred to herein as binding molecules) are molecules that are able to recognize and bind to a specific nucleotide sequence within a target nucleic acid molecule (i.e., the nucleic acid molecule intended to be labeled and/or analyzed). “Sequence specific” when used in the context of a nucleic acid molecule means that the binding molecule recognizes a particular linear arrangement of nucleotides or derivatives thereof.

In some embodiments, the protective compound, i.e. the nucleic acid binding molecule is a protein, a molecular complex, a peptide nucleic acid (PNA), a bisPNA clamp, a pseudocomplementary PNA, a locked nucleic acid (LNA), DNA, RNA, or co-polymers of the above such as DNA-LNA co-polymers. In embodiments in which the protective compound is a nucleic acid or derivative thereof, the linear arrangement preferably includes contiguous nucleotides or derivatives thereof that each bind to a corresponding complementary nucleotide on the nucleic acid-based protective compound. In other embodiments, however, the sequence may not be contiguous as there may be one, two, or more nucleotides that do not have corresponding complementary residues on the protective compound.

Proteins suitable to these analyses may bind to a target nucleic acid molecule in a sequence-specific manner thereby allowing sequence information to be gained from such binding events. These proteins may be DNA or RNA binding proteins, or they may be capable of binding to both DNA and RNA. Examples of such proteins include but are not limited to polymerases such as DNA polymerase including Klenow fragment and reverse transcriptase, an RNA polymerase, a DNA repair enzyme, DNase I, a helicase, nucleases such as restriction endonuclease, a topoisomerase, a ligase, a methylase such as DNA methyltransferase (optionally, engineered to remove methylase activity, but retain scanning ability), DNA repair enzymes and machinery, recombinases and sequence specific transcription factors or repressors such as but not limited to GATA family members, Ikaros, NF-kappaB, SpI, Hox family members, MyoD, fos, jun, NFAT, nuclear hormone receptors, and the like. Virtually any protein (whether having enzymatic activity or not) that is capable of binding to a nucleic acid can be used as a protective compound. An example of a nucleic acid binding agent that binds to single stranded nucleic acids is SPP1-encoded replicative DNA helicase gene 40 product (G40P).

Transposases can also be used to label nucleic acids at discrete sequence sites. Transposases are enzymes involved in moving transposons around in a genome. The sequence specific DNA binding characteristics of the transposons can be exploited according to the invention.

Molecular complexes are complexes of more than one component, i.e., multiple proteins or proteins and oligonucleotides mixed etc. An example of such a complex is RecA filaments which are complexes of RecA protein and oligonucleotides. Such filaments are particularly useful according to the invention because they are capable of specifically blocking large sequences in the DNA.

RecA protein, a recombinase derived from Escherichia coli, is known to catalyze in vitro homologous pairing of single-stranded DNA with double-stranded DNA and thus to generate homologously paired triple-stranded DNA or other triple-stranded joint DNA molecules. RecA protein is also reported to catalyze the formation of a four-stranded DNA structure known as a double D-loop. In this reaction, two types of complimentary single-stranded DNA are used as homologous probes to target double-stranded DNA, which has a homologous site for the single-stranded DNA probe. In addition to DNA-DNA hybridization, RecA protein can also promote RNA-DNA hybridization. For example, single-stranded DNA coated with RecA protein can recognize complimentarity with naked RNA. RecA protein is commercially available from Boehringer-Mannheim, Pharmacia.

RecA-assisted restriction endonuclease (RARE) cleavage is a general and efficient method of targeting restriction enzyme cleavage to unique predetermined sites. This method is based on the ability of RecA to pair oligonucleotides to homologous sequences in duplex DNA to form three-stranded complexes. These complexes protect the selected sites from enzymatic manipulation (e.g., such as methylation or demethylation), and, after removal of the complexes, restriction enzyme cleavage is limited to the selected sites (e.g., unmethylated sites). This method has been used to map and manipulate large segments of DNA.

The invention also encompasses the use of RecA-like recombinases which have catalytic activity similar to native RecA protein. RecA-like recombinases have been isolated and purified from many prokaryotes and eukaryotes. Examples of such recombinases include, but are not limited to, the wild type RecA protein derived from Escherichia coli (Shibata T. et al., Method in Enzymology, 100:197 (1983)), and mutant types of the RecA protein (e.g., RecA 803: Madiraju M. et al., Proc. Natl. Acad. Sci. USA, 85: 6592 (1988); RecA 441(Kawashima H. et al., Mol. Gen. Genet., 193: 288 (1984), etc.); uvsX protein, a T4 phage-derived analogue of the protein (Yonesaki T. et al., Eur. J. Biochem., 148: 127 (1985)); RecA protein derived from Bacillus subtilis (Lovett C. M. et al., J. Biol. Chem., 260: 3305 (1985)); Rec1 protein derived from Ustilago (Kmiec E. B. et al., Cell, 29 :367 (1982)); RecA-like protein derived from heat-resistant bacteria (such as Thermus aquaticus or Thermus thermophilus) (Angov E. et al., J. Bacteriol., 176: 1405 (1994); Kato R. et al., J. Biochem., 114: 926 (1993)); and RecA-like protein derived from yeast, mouse and human (Shinohara A. et al., Nature Genetics, 4: 239 (1993)).

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based tag molecules.

Peptide nucleic acid is synthesized from monomers connected by a peptide bond (Nielsen, P. E. et al. Peptide Nucleic Acids, Protocols and Applications, Norfolk: Horizon Scientific Press, p. 1-19 (1999)). It can be built with standard solid phase peptide synthesis technology.

PNA chemistry and synthesis allows for inclusion of amino acids and polypeptide sequences in the PNA design. For example, lysine residues can be used to introduce positive charges in the PNA backbone, as described below. All chemical approaches available for the modifications of amino acid side chains are directly applicable to PNAs.

PNA has a charge-neutral backbone and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bisPNA, pseudocomplementary PNA (pcPNA).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids Protocols and Applications, Norfolk: Horizon Scientific Press, p. 1-19 (1999)). However, when different concentration ratios are used and/or in the presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize by Hoogsteen pairing. The target sequence can be short (e.g., 8 bp), but the bisPNA/DNA complex is still stable as it forms a hybrid with twice as many (e.g., a 16 bp) base pairings overall. The bisPNA structure further increases specificity of their binding. As an example, binding to an 8 bp site with a tag having a single base mismatch results in a total of 14 bp rather than 16 bp.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diaminopurine (D) instead of adenine and 2-thiouracil (^{s}U) instead of thymine. While D and ^{s}U are still capable of hybridization with T and A respectively, their self-hybridization is sterically prohibited.

Locked nucleic acid (LNA) molecules form hybrids with DNA, which are at least as stable as PNA/DNA hybrids (Braasch, D. A. et al., Chem & Biol. 8(1):1-7(2001)). Therefore, LNA can be used just as PNA molecules would be. LNA binding efficiency can be increased in some embodiments by adding positive charges to it. LNAs have been reported to have increased binding affinity inherently.

In some embodiments, the nucleic acid binding molecule is capable of non-specifically binding and translocating (e.g., “scanning”) along the length of a nucleic acid target. Nucleic acid binding molecules that bind to specific sequences and/or structures (e.g., minor or major groove binding agents) as well as nucleic acid binding molecules that can translocate along the length of a nucleic acid molecule are contemplated.

One example of this technique uses RecA protection and covalent DNA backbone labeling to generate large patches of sequence-specific labeling in the genomic DNA. RecA in combination with oligonucleotides (which form RecA filaments) can be used to site-specifically recognize sequences in genomes. These filaments have been used, for instance, in recA-assisted rare endonuclease (RARE) cleavage (described above) and also protection of restriction sites. The methods of the invention, however, use these RecA filaments in a different manner. For instance, one example involves the following steps: protecting the chosen sequences, from fluorescent labeling, with RecA filaments; fluorescently labeling (e.g., with Cy5 DNA labeling kit from Panvera) the target nucleic acid; removing the RecA filaments and free Cy5 labeling reagent through ethanol precipitation; fluorescently labeling (e.g., with Cy3 Panvera DNA labeling kit) the target nucleic acid; and removing the free Cy3 labeling reagent through ethanol precipitation. The resulting nucleica acid has patches of Cy3 labeling in the regions of interest (i.e., those regions where recA was bound).

The invention also involves methods for protein mapping and kinetic determination using direct, linear DNA analysis. Direct, linear scanning of DNA molecules can be used to map locations of nucleic acid binding proteins on linearized DNA molecules with high accuracy and precision. The mapping of the location of the proteins can be combined with the determination of kinetic binding constants such as on-rate, off-rate, and equilibrium binding constants.

One example involving this type of analysis entails the incubation of a target DNA fragment of interest together with varying concentrations of protein to determine the number of molecules that are bound and not bound to the various sites on the mapped fragment. This is particularly important because for transcription factors and other cis-regulatory binding elements, these may have different binding constants based on different sequence binding sites. This can be used to assess activity at any given locus (e.g., as a measure of gene regulation at a promoter sequence, as a measure of replication, etc.).

Another example involves the co-incubation of the nucleic acid fragment and the protein followed by measurements over a time course and detecting the number of proteins associated with the nucleic acid fragment at different time points.

A third example involves the co-incubation of an excess of competing oligonucleotides followed by measurements of the off-rate for the oligonucleotides or proteins on the nucleic acid.

For the sake of convenience and brevity many of the aspects and embodiments of the invention are referred to solely in terms of DNA. However, it is to be understood that these aspects and embodiments similarly and equally apply to nucleic acids in general and are not limited to DNA, unless otherwise stated.

These methods are a very important set of tools for understanding the complex association of functional elements with promoter, regulatory, enhancer, and other sites on the genome. The real-time nature of the technology allows for the combination of physical map information along with dynamic information, allowing an understanding of the physiological conditions associated with protein binding to a nucleic acid. In some embodiments, the proteins are labeled. In other embodiments, the proteins are not labeled but their pattern of binding (and thus possibly the activity on a given nucleic acid) can still be determined using the blocking compound aspects provided herein.

The proteins may be isolated or in the form of protein extracts, nuclear extracts or cytoplasmic extracts.

The invention also involves methods for mapping transposons using linear analysis. Using linear scanning of DNA, transposons can be mapped in the genome by designing transposon-specific fluorescent tags on the DNA. Transposon mapping using direct, linear analysis may be accomplished, for example through the following steps: isolating the genome of interest containing the transposon; digesting the genome to resolvable sizes to be run through the direct, linear analysis chip; tagging the genome using transposon specific tags (e.g., the tag site can be spliced into the transposon, such as lambda GFP-Cro repressor, or through the design of a novel tag that is unique in the genome of interest); analyzing the sample through the use of the direct, linear analysis chip; and matching the map locations of interest to the genome to determine the location of the transposon.

Thus in one aspect the method identifies a transposon by scanning a nucleic acid sequence comprising at least one labeled transposon with a linear nucleic acid analysis system to identify the transposon.

Transposons are mobile genetic elements that have the ability to translocate to a variety of sites on both chromosomal and extra-chromosomal DNA. Thus, a “transposon” is a segment of DNA that can insert itself into a target DNA at random or at almost random locations. Transposons move (transpose) from a portion of chromosomal DNA, plasmid DNA or viral DNA to another portion of the same or different DNA. They are widely distributed in bacteria, yeasts, maize, Drosophila, etc. The DNA site to which they transpose is not fixed specifically, and it is presumed that they are able to transpose to any DNA site.

Although transposons can be divided into subgroups based on their transposition mechanism, they all have similar DNA element structures (Orle, K. and Craig, N., Gene 1991, 104, 125-131). Transposons in their simplest form carry at least two genes. Typically, one gene codes for an antibiotic resistance factor and the second gene encodes one or more transposases. The transposase is an enzyme responsible for the recognition of the transposon DNA element, the insertion site on the target DNA, and for catalyzing the transposition event.

Mobile genetic elements also carry additional terminal sequence elements that are required for transposition. The two end elements are 10 to 30 base pairs in length and are either identical or closely related sequences that form a pair of terminal inverted repeats. The end elements play at least two functional roles. They act as a sequence specific binding site for the transposase protein and they signal the end of the transposon DNA sequence.

A “transposition reaction” is a reaction wherein a transposon inserts into a target DNA at random or at almost random sites. Essential components in a transposition reaction are a transposon and a transposase or an integrase enzyme or some other components needed to form a functional transposition complex. All transposition systems capable of inserting DNA in a random or in an almost random manner are useful. Examples of natural transposon systems are Ty1 (Devine and Boeke, 1994, and International Patent Application WO 95/23875), Transposon Tn7 (Craig, 1996), Tn.sub.10 and IS10 (Kleckner et al. 1996), Mariner transposase (Lampe et al., 1996), Tc1 (Vos et al., 1996, 10(6), 755-61), Tn5 (Park .et al., 1992), P element (Kaufmnan and Rio, 1992) and Tn3 (Ichikawa and Ohtsubo, 1990), bacterial insertion sequences (Ohtsubo and Sekine, 1996), retroviruses (Varmus and Brown 1989) and retrotransposon of yeast (Boeke, 1989).

The term “transposase” is intended to mean an enzyme capable of forming a functional complex with a transposon or transposons needed in a transposition reaction including integrases from retrotransposons and retroviruses.

A transposition reaction is a three step process that is performed entirely by transposon encoded proteins. The first two steps generate a transposition intermediate and the third step resolves the insertion event. In the first step, the transposon DNA is recognized by a terminal inverted repeat structure and the DNA is cleaved at both ends, generating a pair of 3′-OH termini. Some transposable elements that transpose through a nonreplicative mechanism, such as Tn7, generate double stranded cuts at the ends of the transposon, while transposable elements that transpose through a replicative mechanism, such as phage Mu, generate only a single stranded cut. The second step in the transposition reaction, known as strand transfer, is the concerted cleavage of the target strand DNA coupled with the ligation of the transposon 3-OH groups to the target DNA 5′ phosphates to generate a recombination intermediate. The cleavage of the target DNA and the ligation event do not appear to be energetically coupled in that external sources of ATP are not required. The third transposition step resolves the intermediate recombination structure. The type of processing required is dependent on the type of intermediate created. For the non-replicative elements, gap repair completes the process. In replicative transposition, the strand transfer intermediate is resolved by replication of the transposon, resulting in two copies of the transposon.

An “artificial transposon” is a transposon that is not naturally occurring. Artificial transposons can be easily assembled from a single integration reaction, allowing the recovery of insertions suitably spaced to facilitate DNA analysis. Artificial transposons also can be engineered to contain desired features useful for DNA mapping or sequencing. Other markers can be inserted into the multicloning sites of artificial transposons, including but not limited to yeast and mammalian drug-selectable or auxotrophic genes, generating marker cassettes that can act as transposons. Such artificial transposons can be used for marker addition, i.e., the insertion of a useful auxotrophic marker into an acceptable region of a plasmid of interest.

Transposition is a powerful tool for introducing random or targeted mutations into a genome. Through global transposon mutagenesis and rapid analysis of the samples, it is now possible to correlate genome and organism function to specific genomic regions in a rapid and efficient manner. The methods may be applied using a single transposon or with multiple transposons inserted into the genome. This method will enable the analysis of multiple gene mutations and screening for multi-pathway effects on genome function.

The nucleic acid molecules may be DNA (e.g., genomic DNA), or RNA, or amplification products or intermediates thereof, including complementary DNA (cDNA). The nucleic acid molecules can be directly harvested and isolated from a biological sample (such as a tissue or a cell culture) without the need for prior amplification using techniques such as polymerase chain reaction (PCR).

The sensitivity of methods provided herein allows single nucleic acid molecules to be analyzed individually. The nucleic acid molecules may be single stranded and double stranded nucleic acids. Harvest and isolation of nucleic acid molecules are routinely performed in the art and suitable methods can be found in standard molecular biology textbooks (e.g., such as Maniatis' Handbook of Molecular Biology). DNA includes genomic DNA (such as nuclear DNA and mitochondrial DNA), as well as in some instances cDNA. In important embodiments, the nucleic acid molecule is a genomic nucleic acid molecule. In related embodiments, the nucleic acid molecule is a fragment of a genomic nucleic acid molecule.

In important embodiments of the invention, the nucleic acid molecule is a non in vitro amplified nucleic acid molecule. As used herein, a “non in vitro amplified nucleic acid molecule” refers to a nucleic acid molecule that has not been amplified in vitro using techniques such as polymerase chain reaction or recombinant DNA methods. A non in vitro amplified nucleic acid molecule may however be a nucleic acid molecule that is amplified in vivo (in the biological sample from which it was harvested) as a natural consequence of the development of the cells in vivo. This means that the non in vitro nucleic acid molecule may be one which is amplified in vivo as part of locus amplification, which is commonly observed in some cell types as a result of mutation or cancer development.

The size of the target nucleic acid molecule is not limiting. It can be several nucleotides in length, several hundred, several thousand, or several million nucleotides in length. In some embodiments, the nucleic acid molecule may be the length of a chromosome.

The term “nucleic acid” is used herein to mean multiple nucleotides (i.e. molecules comprising a sugar (e.g. ribose or deoxyribose) linked to an exchangeable organic base, which is either a substituted pyrimidine (e.g. cytosine (C), thymidine (T) or uracil (U)) or a substituted purine (e.g. adenine (A) or guanine (G)). “Nucleic acid” and “nucleic acid molecule” are used interchangeably. As used herein, the terms refer to oligoribonucleotides as well as oligodeoxyribonucleotides. The terms shall also include polynucleosides (i.e. a polynucleotide minus a phosphate) and any other organic base containing polymer. Nucleic acid molecules can be obtained from existing nucleic acid sources (e.g., genomic or cDNA), or by synthetic means (e.g. produced by nucleic acid synthesis).

In some embodiments, it may be desirable to attach a label to the nucleic acid binding molecule and/or the nucleic acid. The label may be attached directly or indirectly and may be covalent or noncovalent. For instance the label may be attached by a bond that can be cleaved under certain conditions. For example, the bond can be one that cleaves under normal physiological conditions or that can be caused to cleave specifically upon application of a stimulus such as light, whereby the agent can be released, leaving only the tag molecule bound to the nucleic acid molecule being labeled or analyzed. Readily cleavable bonds include readily hydrolyzable bonds, for example, ester bonds, amide bonds and Schiff's base-type bonds. Bonds which are cleavable by light are known in the art. Noncovalent methods of conjugation may also be used. Noncovalent conjugation includes hydrophobic interactions, ionic interactions, Van der Waals (or dispersion) interactions, hydrogen bonding, etc. High affinity interactions such as biotin-avidin and biotin-streptavidin complexation, and antigen/hapten-immunoglobulin interactions, and receptor-ligand interactions are also envisioned.

The labels can be detected directly by its ability to emit and/or absorb light of a particular wavelength. A label can be detected indirectly by its ability to bind, recruit and, in some cases, cleave another moiety which itself may emit or absorb light of a particular wavelength. An example of indirect detection is the use of a first enzyme label which cleaves a substrate into visible products. The label may be of a chemical, peptide or nucleic acid nature although it is not so limited.

Generally, the detectable moiety can be selected from the group consisting of an electron spin resonance molecule (such as for example nitroxyl radicals), a fluorescent molecule, a chemiluminescent molecule, a radioisotope, an enzyme substrate, a biotin molecule, a streptavidin molecule, a peptide, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, nucleic acid, a carbohydrate, an antigen, a hapten, an antibody, an antibody fragment, and a lipid.

As used herein, the terms “charge transducing” and “charge transferring” are used interchangeably.

Other detectable labels include radioactive isotopes such as p³² or H³, optical or electron density markers, etc., biotin, digoxigenin, or epitope tags such as the FLAG epitope or the HA epitope, biotin, avidin and enzyme tags such as alkaline phosphatase, horseradish peroxidase, β-galactosidase, etc. Other labels include chemiluminescent substrates, chromogenic substrates, fluorophores such as fluorescein (e.g., fluorescein succinimidyl ester), TRITC, rhodamine, tetramethylrhodamine, R-phycoerythrin, Cy-3, Cy-5, Cy-7, Texas Red, Phar-Red, allophycocyanin (APC), etc. Also envisioned by the invention is the use of semiconductor nanocrystals such as quantum dots, described in U.S. Pat. No. 6,207,392 as labels. Quantum dots are commercially available from Quantum Dot Corporation. The labels (i.e., tags) may be directly linked to the DNA bases or other molecules or may be secondary or tertiary units linked to modified DNA bases.

In some embodiments, the molecules of the invention are labeled with detectable moieties that emit distinguishable signals that are all detected by one type of detection system. For example, the detectable moieties can all be fluorescent labels or radioactive labels. In other embodiments, the molecules are labeled with moieties that are detected using different detection systems. For example, one molecule may be labeled with a fluorophore while another may be labeled with radioactivity.

The label or tag may also be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.). One subset of backbone labels are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS75 1, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

The nucleic acid binding proteins may be detectable. They may be inherently detectable (e.g., auto fluorescing) or extrinsically manipulated to be detectable. In some embodiments, the nucleic acid binding proteins and/or the nucleic acid molecule are labeled with a detectable label. The proteins may be covalently or ionically labeled with the detectable label.

The nucleic acid molecules are analyzed using linear nucleic acid analysis systems. A linear nucleic acid analysis system is a system that analyzes nucleic acids in a linear manner (i.e., starting at one location on the nucleic acid and then proceeding linearly in either direction therefrom). As a nucleic acid is analyzed, the detectable labels attached to it are detected in either a sequential or simultaneous manner. When detected simultaneously, the signals usually form an image of the nucleic acid, from which distances between labels can be determined. When detected sequentially, the signals are viewed in a histogram (signal intensity vs. time), that can then be translated into a map, with knowledge of the velocity of the nucleic acid molecule. It is to be understood that in some embodiments the nucleic acid molecule is attached to a solid support, while in others it is free flowing. In either case, the velocity of the nucleic acid molecule as it moves past, for example, an interaction station or a detector, will aid in determining the position of the labels, relative to each other and relative to other detectable markers that may be present on the nucleic acid molecule.

Accordingly, the linear nucleic acid analysis systems are able to deduce not only the total amount of label on a nucleic acid molecule, but perhaps more importantly, the location of such labels. The ability to locate and position the labels allows these patterns to be superimposed on other genetic maps, in order to orient and/or identify the regions of the genome being analyzed. In preferred embodiments, the linear nucleic acid analysis systems are capable of analyzing nucleic acid molecules individually (i.e., they are single molecule detection systems).

An example of such a system is the Gene Engine™ system described in PCT patent applications WO98/35012 and WO00/09757, published on Aug. 13, 1998, and Feb. 24, 2000, respectively, and in U.S. Pat. No. 6,355,420 B1, issued Mar. 12, 2002. The contents of these applications and patent, as well as those of other applications and patents, and references cited herein are incorporated by reference in their entirety. This system allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide (if one is present) emits a detectable signal. The mechanism for signal emission and detection will depend on the type of label sought to be detected.

Other single molecule nucleic acid analytical methods which involve elongation of a DNA molecule can also be used in the methods of the invention. These include optical mapping (Schwartz, D. C. et al., Science 262(5130):110-114 (1993); Meng, X. et al., Nature Genet. 9(4):432-438 (1995); Jing, J. et al., Proc. Natl. Acad. Sci. USA 95(14):8046-8051 (1998); and Aston, C. et al., Trends Biotechnol. 17(7):297-302 (1999)) and fiber-fluorescence in situ hybridization (fiber-FISH) (Bensimon, A. et al., Science 265(5181):2096-2098 (1997)). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled probe sequences allows determination of sequence landmarks on the nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear nucleic acid analysis systems can also be used, and the invention is not intended to be limited to solely those listed herein.

The nature of such detection systems will depend upon the nature of the detectable moiety used to label the nucleic acid and/or nucleic acid binding proteins, and the like. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

The invention exploits the ability of certain proteins to bind a nucleic acid molecule for labeling and sequencing purposes. Information is gained by analyzing for the presence or absence of a bound nucleic acid binding protein, or by determining the location and relative position of one or more bound proteins. These methods are not dependent upon the nucleic acid molecule being in a linear state. For example, the nucleic acid molecule can be analyzed in a compacted, non-linear state particularly when the only information to be gained is whether or not a protein is bound to a nucleic acid molecule.

The sequence-specific information may be either on a single molecule or on a population of molecules. It is not necessary to label all of the sequence specific sites on a molecule. If there is a homogenous population of molecules then it is possible to partially label members of the population and then reassemble the data to generate a complete map for a particular sequence. This method effectively creates a population of single DNA molecule data with a “nested” set of sequence specific data.

Each nucleic acid molecule so labeled will have a unique pattern of binding by the nucleic acid binding protein. This unique pattern can be akin to a “fingerprint” of the nucleic acid molecule. The greater the number of different nucleic acid binding proteins used (each with a distinguishable detectable signal, whether direct or indirect), the more sequence or activity information is available.

As will be understood based on the foregoing, the methods of the invention can be used to identify nucleic acid regions that are active, as compared to those which are inactive. An active region may be one that is undergoing replication, transcription, modification and the like. An inactive region may be one that is considered “closed” as understood in the art. Such a region may comprise genes that are silent in the cell, as determined by its developmental stage. An understanding and an identification of which genetic regions are “open” and “closed” at certain developmental stages is useful in determining which genes are involved in development, both normal and abnormal. Once such regions have been identified (and including those that are already known based on other methods), then the methods provided herein can also be used to analyze samples from patients, such as biopsy samples to determine the activity of particular loci. Such activity can then be used as a prognostic or diagnostic indicator for the sample and the patient's condition.

Active loci may be associated with or bound to transcription factors, co-factors, polymerases, ligases, recombinases, topoisomerases, cell cycle proteins such as DNA polymerase, cyclins, cyclin dependent kinases, and the like.

Inactive loci may also be associated with or bound to certain proteins or enzymes such as but not limited to methylases, histones, and the like.

The sequencing information derived using the methods of the invention can be compared to genomic sequencing information that is available from sources such as the human genome project. The binding patterns deduced using the methods of the invention can also be superimposed onto physical genomic maps. These maps (including sequence, motif and structural maps) are available from public sources such as the human genome project, or the genome sequencing projects of other organisms. Superimposition of either or both the sequencing information or the binding patterns helps to orient such information and thus identify the region of the genome that is being analyzed. The physical maps of genomes are therefore used as references for orienting the binding patterns determined using the methods of the invention. Moreover, it also helps to identify the genetic loci that are bound. All aspects of the invention may include the step of comparing the binding pattern to a physical map of the genome or part thereof for that particular species.

The genomic maps can be obtained for public databases including the Human Genome Project, the results of which are available from the NCBI or NIH websites. These genomic maps can be sequence maps at various levels of resolution, or they can be motif maps, or structural maps, but they are not so limited.

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

1. A method for identifying a region of a nucleic acid comprising
protecting one or more regions of a nucleic acid with a protective compound,
contacting the protected nucleic acid with a blocking compound to block the non-protected regions of the nucleic acid,
removing the protective compound, and
contacting the nucleic acid with a first label, wherein the first label is detectably distinct from the blocking compound, and
detecting the position of the first label on the nucleic acid to identify the region of the nucleic with a linear nucleic acid analysis system.

2. The method of claim 1, wherein the linear nucleic acid analysis system is a single nucleic acid analysis system.

3. The method of claim 1, wherein the linear nucleic acid analysis system is selected from the group consisting of Gene Engine™, optical mapping, and DNA combing.

4. The method of claim 1, wherein the blocking compound is a second label.

5. The method of claim 4, wherein the second label is a fluorescent label.

6. The method of claim 1, wherein the protective compound is a RecA filament.

7. The method of claim 1, wherein the protective compound is selected from the group consisting of a protein, an oligonucleotide, a peptide nucleic acid (PNA), a locked nucleic acid (LNA), a DNA, an RNA, a bisPNA clamp, a pseudocomplementary PNA, and a LNA-DNA co-polymer.

8. The method of claim 7, wherein the protective compound is an enzyme.

9. The method of claim 8, wherein the enzyme is selected from the group consisting of a DNA polymerase, an RNA polymerase, a DNA repair enzyme, a helicase, a nuclease, a recombinase, and a ligase.

10. The method of claim 1, wherein the first label is a fluorescent label.

11. The method of claim 1, wherein the protective compound binds to the nucleic acid in a sequence non-specific manner.

12. The method of claim 1, wherein the protective compound binds to the nucleic acid in a sequence specific manner.

13. The method of claim 1, wherein the nucleic acid is DNA or RNA.

14. The method of claim 1, wherein the first label is a backbone specific label.

15. The method of claim 1, wherein the linear nucleic acid analysis system comprises exposing the nucleic acid to a station to produce a signal arising from the first label of the nucleic acid, and detecting the signal using a detection system.

16. The method of claim 1, wherein the first label is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

17. A method for determining a property of a nucleic acid-protein interaction, comprising:
contacting a first nucleic acid with a first protein,
determining a first binding interaction between the first nucleic acid and the first protein, and
comparing the first binding interaction with a second binding interaction using a linear nucleic acid analysis system to determine the property of the nucleic acid-protein interaction.

18. The method of claim 17, wherein the second binding interaction involves contacting a second nucleic acid with a second protein, and determining the second binding interaction between the second nucleic acid and the second protein.

19. The method of claim 18, wherein the first and second nucleic acid are identical.

20-32. (canceled)

33. A method for identifying a transposon, comprising:
scanning a nucleic acid comprising at least one labeled transposon with a linear nucleic acid analysis system to identify the transposon.

34-44. (canceled)
